(19) United States
(12) Patent Application Publication
(10) Pub. No.: US 2013/0234071 A1
(43) Pub. Date: Sep. 12, 2013

(54) COMPOSITION OF HUMAN SYNTHETIC URINE

(71) Applicant: Joel David Deneau, Moorpark, CA (US)

(72) Inventor: Joel David Deneau, Moorpark, CA (US)

(21) Appl. No.: 13/417,283

(22) Filed: Mar. 11, 2012

(51) Int. Cl. G01N 33/62

(52) U.S. Cl. CPC G01N 33/62
USPC 252/408.1

(57) ABSTRACT

This invention relates to a novel comprehensive composition comprising the major inorganic salts, urea, organic compounds, organic ammonium salts, proteins and human immuglobins present in normal human urine formulated into unisex synthetic human urine.

### CROSS REFERENCE TO RELATED APPLICATIONS

Non Applicable.

### FEDERALLY SPONSORED RESEARCH OR DEVELOPMENT

Non Applicable.

### REFERENCES DISCLOSED;

U.S. Pat. No. 7,192,776

U.S. Pat. No. 7,011,940

U.S. Pat. No. 6,306,422

U.S. Pat. No. 5,489,281

U.S. Pat. No. 5,489,281

U.S. Pat. No. 4,146,644

U.S. Pat. No. 7,192,776

### BACKGROUND OF THE INVENTION

This invention relates to a novel comprehensive composition comprising the major inorganic salts, urea, organic compounds, organic ammonium salts, proteins and human immuglobins present in normal human urine formulated into unisex synthetic human urine. The use of sodium azide as a preservative solution is also disclosed.

Urine tests are an important and useful diagnostic tool because they are easily obtained and inexpensive. Urine provides important information about a number of physiological processes, including but not limited to renal disease, diabetes mellitus, hydration status, toxicology, and some liver diseases. Many people will have a routine urine examination upon admission to a hospital, and many outpatient settings do routine analyses as well.

Blood is filtered by the kidneys, producing urine to rid the body of wastes and to regulate fluid and electrolyte balance. The normal adult produces between 1.0 to 1.5 liters of urine daily. Enormous amounts of fluids pass through the kidneys every day, as about a thousand liters of blood need to pass through the kidneys to produce one liter of urine.

The nephron is the primary unit of the kidney that produces urine, and each kidney has about one million nephrons. The nephron is composed of the glomerular capsule, the proximal convoluted tubule, the loop of Henle, and the distal convoluted table, which empties into a collecting tubule. Every minute about 125 mL of blood is filtered by the glomerli in an adult male, slightly less in an adult female. The nephrons filter the blood of electrolytes, glucose, water, and small proteins, most of which is reabsorbed in the proximial tubule. Urine in the healthy person contains 96% water and 4% solutes, primarily urea and sodium chloride.

Despite the ease of urine collection, many problems in the handling of specimens occur which will provide inaccurate results. Problems can occur with the collection process, the handling of the urine specimen, or with urine testing. Most urine tests require a minimum of 10 cc of urine. Urine is collected through several methods; catherization, clean-catch, first morning, and pediatric collection.

Urine specimens must be examined within two hours of collection to avoid bacterial contamination. Bacteria that are present within the urine at collection will convert urea to ammonia, giving false alkaline readings. The alkalinity, in turn, leads to breakdown of casts and proteins. To avoid these problems, urine should be immediately refrigerated if it cannot be analyzed within a two hour period.

The disclosed invention in the application is a synthetic urine composition containing all the major analogs of normal unisex human urine.

In response for the need of a reliable source of comprehensively biologically complete standard human urine analog numerous attempts to formulate synthetic urine have been made. For example, U.S. Pat. No. 6,306,422 to Batich et al. (table 3, col. 16, line 50 et seq.), U.S. Pat. No. 5,328,954 to Sarangapani (table 1, col. 9, line 29 et seq.), U.S. Pat. No. 5,489,281 to Watanabe et al. (col. 12, example 6), U.S. Pat. No. 4,146,644 to Griffith et al. (table 1, col. 10), and U.S. Pat. No. 7,192,776. However, none of these references address a composition similar to unisex human urine with all its major constituents.

Further, all the references do not have all the desired composition of human urine and fail to include compounds which are necessary for a comprehensive control for human urine in diagnostic testing methods available to one ordinarily skilled in the art that include but are not limited to human immunoglobins, mammalian derived sources of albumin, inorganic salts, organic compounds, and organic ammonium salts.

The invention disclosed in this application comprises a composition similar to natural human unisex urine. The invention disclosed is useful for all manners of diagnostic testing of human urine where the use of a comprehensive composition is desired.

The invention disclosed in this application can be used in the following including urinalysis, and standard diagnostic testing methods comprising but not limited to, toxicology, urinanlysis, 11-deoxycortisol, 17-hydroxycorticosteriods, 17-ketosteroids, 4-pyrodoxic acid, 5-hydroxyindoleacetic acid, 5-hydroxyproline, addis count, albumin, aldosterone, amino acids, arylsulfate A, Bence Jones Protein, Benedicts test, cyclic adenosine monophosphate, delta-aminolevulinic acid (ALA), dexamethasone suppression, dinitrophenylhydrazine, D-xylose absorption, ferric chloride, glucose, hemosiderin, hydroxyproline, indicant, ketone bodies, melanin, melanocyte-stimulating hormone, mucopolysaccharides qualitiative, nitrate bacteria screen, osmolality, oxalate, phenistix, pregnanetriol, protein electrophoresis, schilling test, specific gravity, thiocyanate, toxicology screening, twenty four hour urine, urethral pressure profile, urinalysis, urine phosphate, urobilinogen, urine phospahate, urobilinogen, vitamins B, water deprivation, water loading and xanthuric acid, and drug screens.

### BRIEF SUMMARY OF THE INVENTION

This invention relates to a novel comprehensive composition comprising the major inorganic salts, urea, organic compounds, organic ammonium salts, proteins and human immunoglobins present in normal human urine formulated into a synthetic unisex human urine (table 1).

The invention disclosed in this application relates to analogs or compositions of a unisex human synthetic urine. The disclosed invention is useful for calibration standards for diagnostic tests, calibration standards for ELISA test, calibration standards for gas chromatography, liquid chromatography, or any other diagnostic test known to one ordinarily skilled in the art of biochemical, metabolite, or diagnostic testing. Further, this invention relates to a comprehensive analog of human synthetic undisclosed in the prior art that is useful as a negative control in all previously disclosed methods of diagnostic testing.

The invention disclosed in the invention can be used in the following methods listed below as negative controls including but not limited to urinalysis, and standard diagnostic testing methods comprising but not limited to, toxicology, urinalysis, 11-deoxycortisol, 17-hydroxycorticosteriods, 17-ketosteroids, 4-pyrodoxic acid, 5-hydroxyindoleacetic acid, 5-hydroxyproline, addis count, albumin, aldosterone, amino acids, arylsulfate A, Bence Jones Protein, Benedicts test, cyclic adenosine monophosphate, delta-aminolevulinic acid (ALA), dexamethasone suppression, dinitrophenylhydrazine, D-xylose absorption, ferric chloride, glucose, hemosiderin, hydroxyproline, indicant, ketone bodies, melanin, melanocyte-stimulating hormone, mucopolysaccharides qualitative, nitrate bacteria screen, osmolality, oxalate, phenistix, pregnanetriol, protein electrophoresis, schilling test, specific gravity, thiocyanate, toxicology screening, twenty four hour urine, urethral pressure profile, urinalysis, urine phosphate, urobilinogen, urine phosphate, urobilinogen, vitamins B, water deprivation, water loading and xanthuric acid and drug screening.

The invention discloses a range of concentrations as a preferred embodiment because natural unisex human urine displays ranges of compositions depending on sex of the individual, metabolism, environmental factors, diet, general health or illness and genetic factors.

The invention comprises a p.H range between 4 and 10.

The invention comprises a specific gravity range between 1.008 and 1.030.

The invention disclosed in this application comprises the preferred embodiments of table 1 of the invention and range of concentrations of the preferred embodiments.

### BRIEF DESCRIPTION OF THE DRAWINGS

FIG. 1 discloses the preferred embodiments comprising the invention of the application in the preferred concentrations.

### DETAILED DESCRIPTION OF THE INVENTION

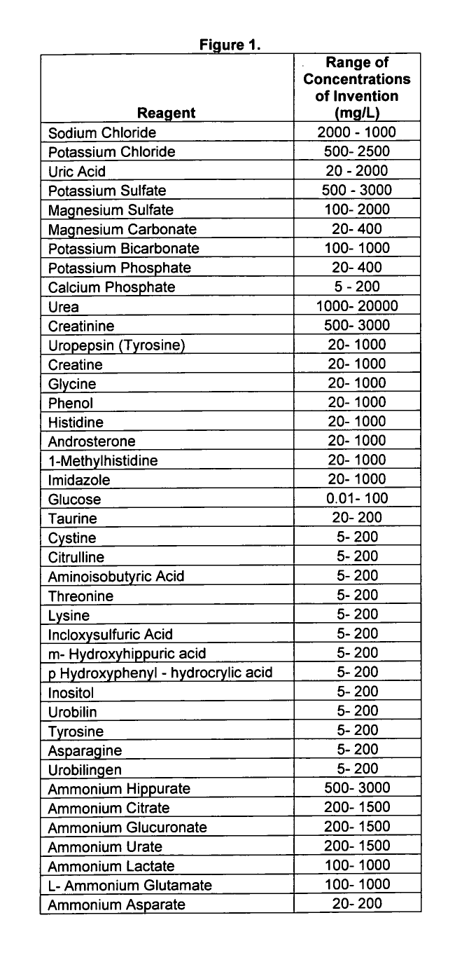

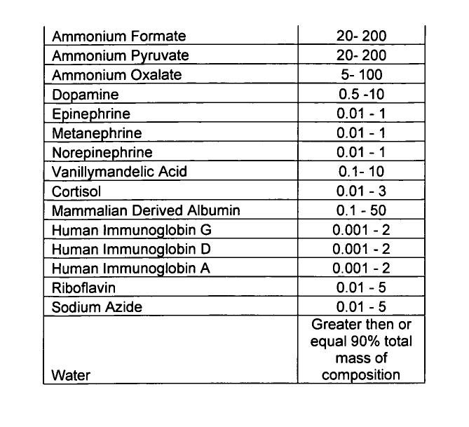

A preferred embodiment of this invention comprises a novel composition comprising the major inorganic salts, urea, organic compounds, organic ammonium salts, proteins and human immunoglobins present in normal human urine formulated into a synthetic urine (FIG. 1).

A preferred embodiment of this invention disclosed comprises analogs or compositions of a unisex human synthetic urine. The disclosed invention is useful for calibration standards for diagnostic tests, calibration standards for ELISA test, calibration standards for gas chromatography, or any other diagnostic test known to one ordinarily skilled in the art of biochemical or metabolite testing. Further, this invention relates to a comprehensive analog of human synthetic undisclosed in the prior art that is useful as a negative control in all previously disclosed methods of diagnostic testing.

A preferred embodiment of this invention is the compositions use in the following methods diagnostic test listed below as a negative control. Such diagnostic testing include but is not limited too toxicology, urinalysis, 11-deoxycortisol, 17-hydroxycorticosteriods, 17-ketosteroids, 4-pyrodoxic acid, 5-hydroxyindoleacetic acid, 5-hydroxyproline, addis count, albumin, aldosterone, amino acids, arylsulfate A, Bence Jones Protein, Benedicts test, cyclic adenosine monophosphate, delta-aminolevulinic acid (ALA), dexamethasone suppression, dinitrophenylhydrazine, D-xylose absorption, ferric chloride, glucose, hemosiderin, hydroxyproline, indicant, ketone bodies, melanin, melanocyte-stimulating hormone, mucopolysaccharides qualitative, nitrate bacteria screen, osmolality, oxalate, phenistix, pregnanetriol, protein electrophoresis, schilling test, specific gravity, thiocyanate, toxicology screening, twenty four hour urine, urethral pressure profile, urinalysis, urine phosphate, urobilinogen, urine phosphate, urobilinogen, vitamins B, water deprivation, water loading and xanthuric acid.

In another embodiment of this invention is it discloses a range of concentrations of the constituents of the composition as a preferred embodiment as natural unisex human urine displays ranges of compositions depending on sex of the individual, metabolism, environmental factors, diet, general health or illness and genetic factors.

In another embodiment the invention disclosed in this application comprises the preferred embodiments of table 1 of the invention and range of concentrations of the preferred embodiments.

In another embodiment the invention comprises the following composition dissolved in reverse osmosis, distilled, tap or natural sources of water; Sodium Chloride (2000 to 10000 mg/L), Potassium Chloride (500-2500 mg/L), Potassium Sulfate (500 to 3000 mg/L), Magnesium Sulfate (100-2000 mg/L), Magnesium Carbonate (20-400 mg/L), Potassium Bicarbonate (100-1000 mg/L), Potassium Phosphate (20-400 mg/L), Calcium Phosphate (5-200 mg/L), Urea (1000-20000 mg/L), Uric Acid (20 2000 mg/L), Creatinine (500-5000 mg/L), Uropepsin (20-1000 mg/L), Creatine (20-1000 mg/L), Glycine (20-1000 mg/L), Phenol (20-1000 mg/L), Histidine (20-1000 mg/L), Androsterone (20-1000 mg/L), 1 Mthylhistidine (20-1000 mg/L), Imidazole (20-1000 mg/L), Glucose (0.01-100), Taurine (20-200 mg/L), Cytosine (5-200 mg/L), Citrulline (5-200 mg/L), Aminoisobutyric Acid (5-200 mg/L), Threonine (5-200 mg/L), Lysine (5-200 mg/L), Incloxysulfuric Acid (5-200 mg/L), m-Hydroxyhippuric Acid (5-200 mg/L), p Hydroxyphenyl [hydrocrylic acid] (5-200 mg/L), Inositol (5-200 mg/L), Urobilin (5-200 mg/L), Tyrosine (5-200 mg/L), Asparagine (5-200 mg/L), Urobilingen (5-200 mg/L), Ammonium Hippurate (500-3000 mg/L), Ammonium Citrate (200-1500 mg/L), Ammonium Glucuronate (200-1500 mg/L), Ammonium Urate (200-1500 mg/L), Ammonium Lactate (100-1000 mg/L), L-Ammonium Glutamate (100-1000 mg/L), Ammonium Asparate (20-200 mg/L), Ammonium Formate (20-200 mg/L), Ammonium Pyruvate (20-200 mg/L), Ammonium Oxalate (5-100 mg/L), Dopamine (0.5-10 mg/L), Epinephrine (0.01-1 mg/L), Metanephrine (0.01-1 mg/L), Norepinephrine (0.01-1 mg/L), Vanillymandelic Acid (0.1 to 10 mg/L), Cortisol (0.01-3 mg/L), Mammalian Derived Albumin (0.1-50mg/L), Human Immunoglobin G (0.0001-1 mg/L), Human Immunoglobin D (0.0001-1 mg/L), Human Immunglobin A (0.0001-1 mg/L), Riboflavin (0.01-10 mg/L), Sodium Azide (0.1-10 mg/L), and reverse osmosis, distilled, tap or natural sources of water at a concentration greater or equal to 90% of the total mass of the composition.

In another embodiment the invention comprises a p.H range between 4 and 10.

In another embodiment the invention comprises a specific gravity range between 1.008 and 1.030.

1. (canceled)

2. (canceled)

3. (canceled)

4. (canceled)

5. (canceled)

6. (canceled)

7. The sterile composition comprising; Sodium Chloride, Potassium Chloride, Potassium Sulfate, Magnesium Sulfate, Magnesium Carbonate, Potassium Bicarbonate, Potassium Phosphate, Calcium Phosphate, Urea, Uric Acid, Creatinine, Uropepsin, Creatine, Glycine, Phenol, Histidine, Androsterone, 1 Methylhistidine, Imidazole, Glucose, Taurine, Cytosine, Citrulline, Aminoisobutyric Acid, Threonine, Lysine, Incloxysulfuric Acid, m-Hydroxyhippuric Acid, p Hydroxyphenyl [hydrocrylic acid], Inositol, Urobilin, Tyrosine, Asparagine, Urobilingen, Ammonium Hippurate, Ammonium Citrate, Ammonium Glucuronate, Ammonium Urate, Ammonium Lactate, L-Ammonium Glutamate, Ammonium Asparate, Ammonium Formate, Ammonium Pyruvate, Ammonium Oxalate, Dopamine, Epinephrine, Metanephrine, Norepinephrine, Vanillymandelic Acid, Cortisol, Mammalian Derived Albumin, Human Immunoglobin G, Human Immunoglobin D, Human Immunglobin A, Dye Agent, gallic acid, antimicrobial agent dissolved in water wherein solution is further sterilized by exposure to ultra violet light, radiation, ethylene oxide treatment, ozone treatment, colloidal silver, radiation, filtration, heat, or any other method of sterilization.

8. The composition of claim 1 comprising a pH range between 4 and 10.

9. The composition of claim 1 comprising a specific gravity range between 1.008 g/cm̂3 and 1.030 g/cm̂3.

10. The sterile composition comprising; Sodium Chloride, Potassium Chloride, Potassium Sulfate, Magnesium Sulfate, Magnesium Carbonate, Potassium Bicarbonate, Potassium Phosphate, Calcium Phosphate, Urea, Uric Acid, Creatinine, Uropepsin, Creatine, Glycine, Phenol, Histidine, Androsterone, 1 Methylhistidine, Imidazole, Glucose, Taurine, Cytosine, Citrulline, Aminoisobutyric Acid, Threonine, Lysine, Incloxysulfuric Acid, m-Hydroxyhippuric Acid, p Hydroxyphenyl [hydrocrylic acid], Inositol, Urobilin, Tyrosine, Asparagine, Urobilingen, Ammonium Hippurate, Ammonium Citrate, Ammonium Glucuronate, Ammonium Urate, Ammonium Lactate, L-Ammonium Glutamate, Ammonium Asparate, Ammonium Formate, Ammonium Pyruvate, Ammonium Oxalate, Dopamine, Epinephrine, Metanephrine, Norepinephrine, Vanillymandelic Acid, Cortisol, Mammalian Derived Albumin, Human Immunoglobin G, Human Immunoglobin D, Human Immunglobin A, Dye Agent, gallic acid, antimicrobial agent not dissolved in water wherein composition is further sterilized by exposure to ultra violet light, radiation, ethylene oxide treatment, ozone treatment, colloidal silver, radiation, filtration, heat, or any other method of sterilization.

11. The composition of claim 1 further comprising gonadotropins, follicle stimulating hormone, lutenizing hormone, estrone, estradial, estriol, 2-Hydroxyestrogens,1,6α-Hydroxyestrone, 4-Hydroxyestrone, 2-Methoxyestrone, 2-Methoxyestradiol, Pregnanediol, Testosterone, Androsterone, Etiocholanolone, Pregnanetriol, Cortisone, Cortisol, THE, 5a-THF, THF, l lb-hydroxyandrosterone, 11 b-hydroxyetiocholanolone, THB, THA, 5a-androstanediol, 5b-androstanediol, 5a-THB, Aldosterone, and human growth hormone.

12. The composition of claim 1 further comprising human biological matter including but not limited to mammalian red blood cells, mammalian white blood cells (leukocytes) and mammalian epithelial cells.

13. The composition of claim 1 further comprising mammalian hyaline, mammalian waxy, mammalian granular, and mammalian red cell casts and mammalian white blood cell casts.

14. The composition of claim 7 further comprising human biological matter including but not limited to red blood cells, white blood cells (leukocytes) and epithelial cells.

15. The composition of claim 7 further comprising hyaline, waxy, granular, and red and white blood cell casts.

16. The composition of claim 7 further comprising gonadotropins, follicle stimulating hormone, lutenizing hormone, estrone, estradial, estriol, 2-Hydroxyestrogens,1,6α-Hydroxyestrone, 4-Hydroxyestrone, 2-Methoxyestrone, 2-Methoxyestradiol, Pregnanediol, Testosterone, Androsterone, Etiocholanolone, Pregnanetriol, Cortisone, Cortisol, THE, 5a-THF, THF, 11b-hydroxyandrosterone, 11b-hydroxyetiocholanolone, THB, THA, 5a-androstanediol, 5b-androstanediol, 5a-THB, Aldosterone, and human growth hormone.

17. The composition of claim 7 comprising a pH range between 4 and 10.

18. The composition of claim 7 comprising a specific gravity range between 1.008 g/cm̂3 and 1.030 g/cm̂3.

19. The composition of claim 1 wherein said composition is sterilized by means of aseptic processing including but not limited to, exposure to ultra violet light, radiation, ethylene oxide treatment, ozone treatment, colloidal silver, radiation, filtration, heat, or any other methods of sterilization that is known in the art.

20. The composition of claim 7 wherein said composition is sterilized by means of aseptic processing including but not limited to, exposure to ultra violet light, radiation, ethylene oxide treatment, ozone treatment, colloidal silver, radiation, filtration, heat, or any other methods of sterilization that is known in the art.

21. The composition of claim 1 wherein said composition further comprises urobilin.
